(19) United States
(12) Patent Application Publication
(10) Pub. No.: US 2011/0071513 A1
(43) Pub. Date: Mar. 24, 2011

(54) COMMON-MODE NOISE FILTER OF RADIO FREQUENCY GENERATOR FOR RADIOFREQUENCY ABLATION

(75) Inventors: Kyong-Min SHIN, Seoul (KR); Hong-chea Jung, Gimpo-Si (KR); Young-jin Choi, Seoul (KR); Jun-hyok Lee, Kimpo-si (KR); Jae-Bok Kim, Suwon-si (KR); Guk-joo Na, Kimpo-Si (KR); Kye-joo Kim, Kimpo-Si (KR)

(73) Assignees: Taewoong Medical Co., Ltd., Kimpo-Si (KR); Kyong Min SHIN, Seoul (KR)

(21) Appl. No.: 12/730,584

(22) Filed: Mar. 24, 2010

(30) Foreign Application Priority Data
Sep. 23, 2009 (KR) 10-2009-0090124

(51) Int. Cl. A61B 18/18

(52) U.S. Cl. 606/33

(57) ABSTRACT

Disclosed herein is a common-mode noise filter of an RF generator for radiofrequency ablation. The common-mode noise filter of the RF generator for RFA amplifies an oscillation frequency provided by an oscillator to AC output using amplification means, and providing the amplified oscillation frequency to electrodes. The common-mode noise filter is disposed between the amplification means and the electrodes, thus eliminating harmonic components generated by the electrodes depending on conditions of medical treatment when the electrodes are used. Accordingly, the present invention can prevent the distortion of images or waveforms attributable to harmonic components from occurring in a diagnostic imaging device such as a diagnostic ultrasound system used together with the RF generator for RFA as well as in the RF generator's own display unit.

### BACKGROUND OF THE INVENTION

1. Field of the Invention

The present invention relates, in general, to the common-mode noise filter of a radio frequency (RF) generator for radiofrequency ablation (RFA). More particularly, the present invention relates to the common-mode noise filter of an RF generator for RFA, which can eliminate harmonic components that increase depending on conditions of medical treatment when an electrode is used.

2. Description of the Related Art

Generally, as methods of treating cancerous tissue generated in the bodily organs of a human being, for example, the liver, there are methods of suppressing the growth of cancerous tissue and eliminating the cancerous tissue using drugs and radiotherapy without performing surgery, and methods of surgically eliminating cancerous tissue by performing surgery.

Of the above-described treatment methods, the methods of surgically eliminating cancerous tissue are disadvantageous in that, since a region around a lesion at which cancerous tissue is located must be excised, the region to be excised is very wide, so that the surgery itself is very difficult, and much time is required for a region operated on to recover completely, and, in addition, a large scar remains on the region after recovery.

In particular, cancerous tissue or the like frequently recurs, and, when this happens, the region around the previously excised lesion must be excised again, thus not only inflicting pain because of the surgery putting the patient at high risk, but also imposing an economic burden.

Accordingly, recently, methods of eliminating cancerous tissue without performing surgery, that is, methods such as transarterial chemoembolization, percutaneous ethanol injection (PEI), systemic chemotherapy, and local thermal therapy, have been widely performed. Among these methods, local thermal therapy is the most effective method of the methods which are widely performed.

The above-described local thermal therapy includes radiofrequency ablation (RFA), microwave ablation, laser ablation, etc. Among these methods, RFA is the most effective method. Such RFA is a method of ablating and destroying only cancerous tissue using RF heat without excising the cancerous tissue when it occurs in a bodily organ, for example, the liver.

In accordance with an embodiment, an apparatus for removing cancerous tissue (cells) by means of RFA as described above may include an RF generator for radiofrequency ablation which generates a radio frequency of a predetermined level, and a single electrode to which the radio frequency generated by the RF generator for RFA is applied.

Meanwhile, the ‘multi-RF generator for radiofrequency ablation’ disclosed in Korean Patent No. 10-739002, which was filed and registered, proposes a technology which not only can extend the range of ablation, but also can improve on safety while efficiently destroying a large-sized cancer tumor by providing radio frequencies from a single RF generator for RFA to a plurality of electrodes, and which enables RFA to be simultaneously performed on two lesions by controlling one or more channels.

However, the RF generator for RFA is a device which is capable of heating and destroying cancerous tissue in places such as the liver or the thyroid gland through an electrode at the time of performing surgery on the cancerous tissue with high power of 30 to 200 W at a frequency of 480 kHz, and which causes electromagnetic interference with surrounding medical instruments. In particular, a diagnostic imaging device such as a diagnostic ultrasound system for providing images of a region being operated on to a medical team is used together with the RF generator for RFA. Such a diagnostic imaging device is a device sensitive to electromagnetic waves, and may occasionally not provide clear images to the medical team due to electromagnetic waves generated by the RF generator for RFA.

The ultrasound probe of the diagnostic ultrasound system uses a frequency band of 4 MHz to 13 MHz according to the purpose thereof. Therefore, since 480 kHz harmonics (2nd, 3rd, . . . , Nth harmonics) generated by the RF generator for RFA are distributed in a frequency band used by the diagnostic ultrasound system, there is a problem in that the harmonics cause interference with diagnostic images.

Of course, since the harmonics also influence waveforms displayed on the display unit of the RF generator for RFA, there is a problem in that a medical team cannot accurately monitor waveforms or images on the display unit.

### SUMMARY OF THE INVENTION

Accordingly, the present invention has been made keeping in mind the above problems occurring in the prior art, and an object of the present invention is to provide the common-mode noise filter of an RF generator for RFA, which can eliminate harmonic components occurring depending on the conditions of medical treatment when an electrode is used, thus preventing the distortion of images or waveforms attributable to interference in a diagnostic imaging device such as a diagnostic ultrasound system as well as in the RF generator itself for RFA.

In order to accomplish the above object, the present invention provides a common-mode noise filter of a radio frequency (RF) generator for radiofrequency ablation (RFA), the common-mode noise filter amplifying an oscillation frequency provided by an oscillator to AC output using amplification means, and providing the amplified oscillation frequency to electrodes, wherein the common-mode noise filter is disposed between the amplification means and the electrodes, thus eliminating harmonic components generated by the electrodes depending on conditions of medical treatment when the electrodes are used.

Preferably, the common-mode noise filter comprises a transformer, first ends of primary and secondary coils of which are respectively connected to output terminals of the amplification means and second ends of the primary and secondary coils of which are respectively connected to the electrodes, and a capacitor connected in parallel with the transformer.

### BRIEF DESCRIPTION OF THE DRAWINGS

The above and other objects, features and advantages of the present invention will be more clearly understood from the following detailed description taken in conjunction with the accompanying drawings, in which:

FIG. 1 is a diagram showing the common-mode noise filter of an RF generator for RFA according to the present invention; and

FIG. 2 is a graph showing the generation of a sine wave performed by filtering noise using the common-mode noise filter of the RF generator for RFA according to the present invention.

### DESCRIPTION OF THE PREFERRED EMBODIMENTS

Hereinafter, embodiments of the present invention will be described in detail with reference to the attached drawings.

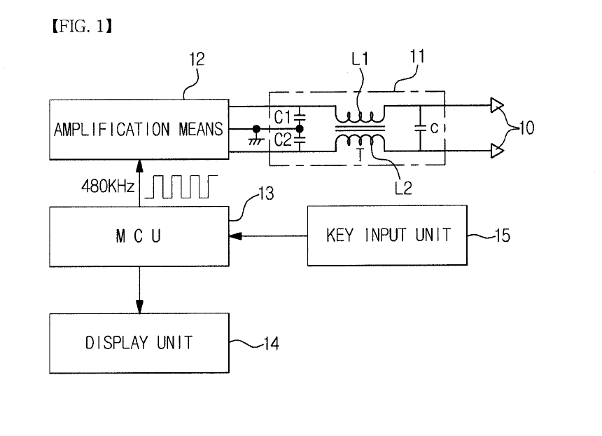

FIG. 1 is a diagram showing the common-mode noise filter of a radio frequency (RF) generator for radiofrequency ablation (RFA) according to the present invention. The present invention is configured such that, when Alternating Current (AC) power amplified by an amplification means 12 is supplied to electrodes 10 under the control of a microcontroller (MCU) which is provided with an oscillation frequency output from an oscillator (not shown) or the like, harmonic components generated by the electrodes 10 depending on the conditions of medical treatment are eliminated by a common-mode noise filter 11, thus preventing interference attributable to electromagnetic waves from occurring in the RF generator's own display unit 14 or another diagnostic imaging device such as a diagnostic ultrasound system.

The MCU 13 for controlling the overall operation of the RF generator for RFA is connected to a key input unit 15, the display unit 14 and the amplification means 12. The key input unit 15 includes keys required to set the operation mode or the like. The display unit 14 displays waveforms or images indicating operating states. The amplification means 12 receives high voltage (HV) and amplifies the oscillation frequency to Alternating Current (AC) power under the control of the MCU 13. The amplification means 12 includes a phase control unit, an amplifier, a matching network, switching elements, various types of sensors, etc.

The common-mode noise filter 11, which eliminates harmonic components generated by the electrodes 10 depending on the conditions of medical treatment when the electrodes 10 are used, are disposed between the amplification means 12 and the electrodes 10. The common-mode noise filter 11 includes a transformer T in which the first ends of respective coils L1 and L2 are connected to the output terminal of the amplification means 12, and the second ends of the coils L1 and L2 are connected to the electrodes 10, and a capacitor C which is connected in parallel with the transformer T on a load side.

Prior to the description of the operation of the present invention, the term “common mode” will be described.

Sunlight has a relative brightness estimated at a specific location, that is, the location on the earth, and separate external energy is not provided to the sun, and thus only energy in common mode is present. In other words, since the same amount of energy is radiated to the outside at the same time with the same phase, such a mode is designated as “common mode”.

For example, when an incandescent light bulb is turned on by supplying commercial power of 60 Hz, the 60 Hz commercial power provides energy through a line (hot) for supplying electricity, and forms a closed circuit through a feedback line (neu). An incandescent light bulb receives 60 Hz commercial power, generates energy in an entirely new frequency band, that is, light, and radiates the same amount of energy to the outside in the same phase at the same time.

In this case, differential mode (normal mode) and common mode coexist. Components in the differential mode denote the voltages of signal/noise appearing between wires as in the case of 60 Hz commercial power. In the case of the common mode, only 60 Hz commercial power is supplied through an input terminal, but signal energy in a new frequency band generated by a working load has an energy level higher than that of the ground surface in which the load is a comparison target.

Therefore, new energy generated by the load has current components intending to move outside the load. In this case, the current components are implemented in common mode in which they are identical in magnitude, phase, time and direction. Of course, common mode signal/noise denotes an energy level relative to the ground surface which is a comparison target.

Common mode signal/noise has the characteristics of being propagated much farther. Electromagnetic waves spatially radiated from a certain RF transmitter have common mode W components, whereas energy by which spatial RF waves are induced to a cable, an antenna, or a specific device also corresponds to common mode. That is, such common mode signal/noise indicates the energy intensity relative to the ground surface.

Contrary to common mode, differential mode is a form in which energy is transmitted using reciprocating wires. That is, the form of energy transmission of 60 Hz commercial power corresponds to the differential mode.

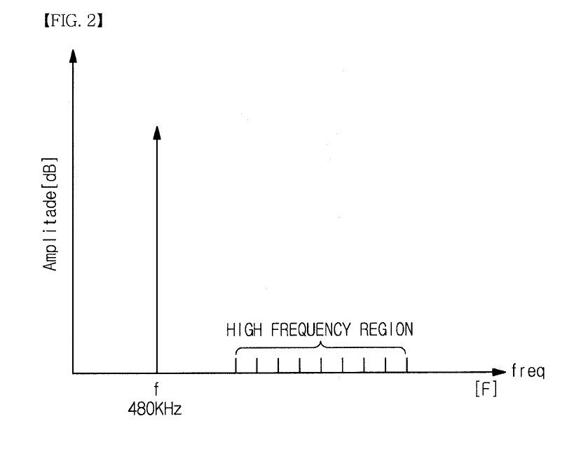

The amplification means 12 in FIGS. 1 and 2 amplifies an oscillation frequency of 480 kHz to high voltage (HV) and supplies the amplified oscillation frequency to the electrodes 10 under the control of the MCU 13 according to the operating environment set by the key input unit 15.

In this case, common mode noise, that is, the harmonic components of AC output, are caused depending on the conditions of the medical treatment when the electrodes 10 are used. These harmonic components are blocked by the common-mode noise filter 11 including the transformer T and the capacitors C1 and C2 connected to the output terminal of the amplification means 12, thus preventing the display unit 14 or a diagnostic imaging device such as a diagnostic ultrasound system located near the RF generator from being influenced.

Therefore, according to the present invention, there are advantages in that harmonic components occurring depending on the conditions of medical treatment when an electrode is used are eliminated by a common-mode noise filter, thus preventing the distortion of images or waveforms attributable to interference in a diagnostic imaging device such as a diagnostic ultrasound system as well as in an RF generator itself for RFA.

Although the preferred embodiments of the present invention have been disclosed for illustrative purposes, those skilled in the art will appreciate that various modifications, additions and substitutions are possible, without departing from the scope and spirit of the invention as disclosed in the accompanying claims.

What is claimed is:

1. A common-mode noise filter of a radio frequency (RF) generator for radiofrequency ablation (RFA), the common-mode noise filter amplifying an oscillation frequency provided by an oscillator to AC output using amplification means, and providing the amplified oscillation frequency to electrodes, wherein:
the common-mode noise filter is disposed between the amplification means and the electrodes, thus eliminating harmonic components generated by the electrodes depending on conditions of medical treatment when the electrodes are used.

2. The common-mode noise filter according to claim 1, comprising:
a transformer, first ends of primary and secondary coils of which are respectively connected to output terminals of the amplification means and second ends of the primary and secondary coils of which are respectively connected to the electrodes; and
a capacitor connected in parallel with the transformer.
